(19) United States
(12) Patent Application Publication
(10) Pub. No.: US 2006/0293294 A1
(43) Pub. Date: Dec. 28, 2006

(54) Method for treatment or prevention of androgen deficiency

(75) Inventors: Taru Blom, Nousiainen (FI); Janne Komi, Turku (FI); Risto Lammintausta, Turku (FI)

Correspondence Address: FITZPATRICK CELLA HARPER & SCINTO, 30 ROCKEFELLER PLAZA, NEW YORK, NY, 10112, US

(73) Assignee: HORMOS MEDICAL CORPORATION, Turku (FI)

(21) Appl. No.: 11/183,185

(22) Filed: Jul. 18, 2005

Related U.S. Application Data
(60) Provisional application No. 60/606,907, filed on Sep. 3, 2004.

(51) Int. Cl. A61K 31/56; A61K 31/4164; A61K 31/138

(52) U.S. Cl. 514/171; 514/649; 514/651; 514/396

(57) ABSTRACT

This invention relates to a method for the treatment or prevention of androgen deficiency in a male individual by administering to the individual an effective amount of a selective estrogen receptor modulator, or an isomer, isomer mixture, metabolite or a pharmaceutically acceptable salt thereof. Furthermore, the invention concerns methods for the treatment or prevention of diseases or disorders caused by androgen deficiency.

### CROSS REFERENCE TO RELATED APPLICATION

The present application is related to and claims priority under 35 U.S.C. §119(e) to U.S. provisional patent application Ser. No. 60/606,907, filed Sep. 3, 2004, incorporated herein by reference.

### FIELD OF THE INVENTION

This invention relates to a method for treatment or prevention of androgen deficiency in a male individual, said method comprising administering to the individual an effective amount of a selective estrogen receptor modulator (SERM). The invention concerns further methods for treatment or prevention of diseases or disorders caused by said androgen deficiency.

### BACKGROUND OF THE INVENTION

The publications and other materials used herein to illuminate the background of the invention, and in particular, cases to provide additional details respecting the practice, are incorporated by reference.

Testosterone in Men

Masculine sex hormones, the androgens, are responsible for the development of the masculine sex characteristics. Furthermore, they are required for reproduction. The main element of the androgens is testosterone, which is imperative for the development and the function of the internal and external masculine sex organs, which has a supportive influence regarding muscle growth, which determines the distribution and the density of hair growth, which has a positive influence with respect to the production of erythrocytes and with respect to the distribution of erythropoietin and the cognitive functions. A shortage of testosterone (hypogonadism) may be classified into two principle forms, which are designated primary and secondary hypogonadism. Diseases based on testosterone shortage include for instance osteoporosis, muscle atrophy, senescence outfall symptoms, the decrease of libido and potency, depression and anaemia.

Primary Hypogonadism

The lack of testosterone production or a decreased testosterone production within the body, originating from a malfunction of the testicles, which is the main synthesis location of testosterone, is designated primary hypogonadism.

Primary hypogonadism includes the testicular failure due to congenital or acquired anorchia, XYY Syndrome, XX males, Noonan's Syndrome, gonadal dysgenesis, Leydig cell tumors, maldescended testes, varicocele, Sertoli-Cell-Only Syndrome, cryptorchidism, bilateral torsion, vanishing testis syndrome, orchiectomy, Klinefelter's Syndrome, chemotherapy, toxic damage from alcohol or heavy metals, and general disease (renal failure, liver cirrhosis, diabetes, myotonia dystrophica). Patients with primary hypogonadism show an intact feedback mechanism in that the low serum testosterone concentrations are associated with high FSH (follicle-stimulating hormone) and LH (luteinizing hormone) concentrations. However, because of testicular or other failures, the high LH concentrations are not effective at stimulating testosterone production.

Secondary Hypogonadism

If the main reason for the diseases is a malfunction of the hypothalamus or the hypophysis the disease is named secondary (or hypogonadotrophic) hypogonadism. This involves an idiopathic gonadotropin or LH-releasing hormone deficiency. This type of hypogonadism includes Kallman's Syndrome, Prader-Labhart-Willi's Syndrome, Laurence-Moon-Biedl's Syndrome, pituitary insufficiency/adenomas, Pasqualini's Syndrome, hemochromatosis, hyperprolactinemia, or pituitary-hypothalamic injury from tumors, trauma, radiation, or obesity. Because patients with secondary hypogonadism do not demonstrate an intact feedback pathway, the lower testosterone concentrations are not associated with increased LH or FSH levels. Thus, these men have low testosterone serum levels but have gonadotropins in the normal to low range.

Age-Related Testosterone Deficiency

Men experience a slow but continuous decline in average serum testosterone after approximately age 20 to 30 years. Researchers estimate that the decline is about 1-2% per year. Moreover, as men age, the circadian rhythm of testosterone concentration is often muted, dampened, or completely lost. The untreated testosterone deficiency in older men may lead to a variety of physiological changes, including sexual dysfunction, decreased libido, loss of muscle mass, decreased bone density, depressed mood, and decreased cognitive function. The net result is male andropause, also known as late-onset hypogonadism or androgen decline in the ageing male (ADAM).

Diagnosis and Treatment of Testosterone Deficiency

The normal ranges for testosterone concentration vary as well as the definition of the limit value to diagnose hypogonadism. The report of the Endocrine Society's Second Annual Andropause Consensus Meeting (Endocrine Society, 2002) delineated three categories for consideration in screening and diagnosing hypogonadism in men over 50 years of age: 1) if total testosterone is ≦200 ng/dL (i.e., 7 nmol/L), diagnosis of androgen deficiency is confirmed; serious hypothalamic or pituitary disease in men with hypogonadotropic hypogonadism to be ruled out; 2) if total testosterone levels are between 200 and 400 ng/dL (i.e., 7-14 nmol/L), additional measures of testosterone and further evaluation before considering testosterone therapy are recommended; and 3) if total testosterone levels are >400 ng/dL (i.e., 14 nmol/L), there is no testosterone deficiency. Many studies have used the 300 to 350 ng/dL (i.e., 10-12 nmol/L) range of total testosterone as a cutoff for identifying hypogonadal patients (in Testosterone and Aging, Clinical Research Directions 2004, ed. Liverman C T and Blaxer D G). In addition to the low testosterone serum concent ration, sign(s) and/or symptom(s) of testosterone deficiency should be present for the diagnosis.

The treatment is usually a substitution therapy which effectively can be measured directly based on the testosterone concentration in serum. The aim of the testosterone substitution is to increase the testosterone concentration in serum to the normal value. Currently, testosterone/androgen compounds are used as treatments for hypogonadism.

Selective Estrogen Receptor Modulators

“SERM”s (selective estrogen receptor modulators) have both estrogen-like and antiestrogenic properties (Kauffmnan & Bryant, Drug News Perspect 8:531-539, 1995). The effects may be tissue-specific as in the case of tamoxifen and toremifene which have estrogen-like effects in the bone, partial estrogen-like effect in the uterus and liver, and pure antiestrogenic effect in breast cancer. Raloxifene and droloxifen are similar to tamoxifen and toremifene, except that their antiestrogenic properties dominate. They are known to decrease total and LDL cholesterol, thus deminishing the risk of cardiovascular diseases, and they may prevent osteoporosis and inhibit breast cancer growth in postmenopausal women.

A review of investigated and/or marketed SERM compounds has been published by V. Craig Jordan (J Medicinal Chemistry 46(7):1081-1111, 2003), incorporated herein by reference.

### SUMMARY OF THE INVENTION

The inventors of the present invention have surprisingly found that compounds belonging to the group of selective estrogen receptor modulators are effective in raising the serum testosterone level in men.

Thus, this invention concerns a method for treatment or prevention of androgen deficiency in a male individual, said method comprising administering to the individual an effective amount of a selective estrogen receptor modulator, or an isomer, isomer mixture, metabolite or a pharmaceutically acceptable salt thereof.

Furthermore, this invention concerns a method for prevention or treatment of a disease or disorder in a male individual, said disease or disorder being caused by androgen deficiency in said individual, said method comprising administering to the individual an effective amount of a selective estrogen receptor modulator as defined in any of the claims 1-6, or an isomer, isomer mixture, metabolite or a pharmaceutically acceptable salt thereof.

### BRIEF DESCRIPTION OF THE DRAWING

FIG. 1 shows serum concentration of testosterone in men versus time during treatment with different doses of fispemifene.

### DETAILED DESCRIPTION OF THE INVENTION

Definitions

The term “treatment” or “treating” shall be understood to include complete curing of a disease or disorder, amelioration or alleviation and of said disease or disorder and delaying the progress or onset of said disease or disorder.

The term “prevention” shall be understood to include complete prevention, prophylaxis, as well as lowering the individual's risk of falling ill with said disease or disorder.

The term “individual” refers to a human or animal subject.

The expression “effective amount” is meant to include any amount of an agent according to the present invention that is sufficient to bring about a desired therapeutical result, especially upon administration to an animal or human subject.

The term “androgen deficiency” shall mean a condition in the male individual where the serum level of masculine sex hormones, particularly testosterone and dihydrotestosterone, is decreased.

The term “testosterone deficiency” refers to a condition in the male individual where the serum level of testosterone is decreased, particularly decreased to a serum level below or at the lower range of the normal reference level. The reference level depends on the laboratory methods used.

The wording “selective estrogen receptor modulator” and any specific compound belonging to this group shall be understood to cover any geometric isomer, any stereoisomer, racemate or other mixture of isomers of the compound. Furthermore, pharmaceutically acceptable salts and other derivatives such as esters as well as metabolites are also included.

Diseases or Disorders Which can be Prevented or Treated by Treating or Preventing Androgen Deficiency using SERMs

In accordance with the present invention, SERMs are useful for prevention or treatment of any disease or disorder in male individual, said disease or disorder being caused by androgen deficiency.

Hypogonadism, particularly secondary hypogonadism, and age-related testosterone deficiency are examples of disorders which can treated or prevented by administrating SERMs according to this invention. Also specific diseases or disorders resulting from said hypogonadism or age-related testosterone deficiency can be treated or prevented. However, also other diseases or disorders which are caused by androgen deficiency but which are unrelated to hypogonadism or age-related testosterone deficiency may be treated or prevented according to the method of this invention.

Thus, as examples of specific diseases or disorder which can be treated or prevented according to the present invention can be mentioned:

hypogonadism, particularly but not restricted to secondary hypogonadism resulting from diseases or disorders such as Kallman's Syndrome, Prader-Labhart-Willi's Syndrome, Laurence-Moon-Biedl's Syndrome, pituitary insufficiency/adenomas, Pasqualini's Syndrome, hemochromatosis, hyperprolactinemia, pituitary-hypothalamic injury from tumors, trauma, radiation, obesity, chronic illness, such as diabetes mellitus, hypotyroidism or other disease or disorder that may affect central production of gonadotropin;

age-related testosterone deficiency and diseases or disorders resulting therefrom, such as impaired muscle strength, sexual dysfunction, decreased libido, loss of muscle mass, decreased bone density, depressed mood, and decreased cognitive function; and

any muscular atrophy/dystrophies; lipodistrophy; long-term critical illness; sarcopenia; frailty or age-related functional decline; reduced muscle strength and function; muscle wasting from HIV; chronic renal failure, reduced bone density or growth; catabolic side effects of glucocorticoids; chronic fatigue syndrome; reduced bone fracture repair; acute fatigue syndrome and muscle loss following elective surgery; cachesia; chronic catabolic state; eating disorders; side effects of chemotherapy; wasting; depression; nervousness irritability; stress; growth retardation; senescence outfall symptoms; reduced cognitive function; anaemia; male contraception; infertility; Syndrome X; diabetic complications or obesity.

SERMs increasing testosterone have the potential to provide novel treatments for male andropause, also known as late-onset hypogonadism or androgen decline in the aging male (ADAM), and both osteoporosis and sexual dysfunction in both men and women. Testosterone deficiency in older men may impair muscle strength (causing frailty/disability); cognitive or sexual function; or vitality/well-being/quality of life.

Advantages of SERMs in the Treatment of Androgen Deficiency

A SERM increasing testosterone sufficiently to treat testosterone deficiency may have several advantages over direct testosterone substitution. The benefits of the increased testosterone can be achieved while a SERM compound, due to its anti-estrogenic or estrogenic effects, simultaneously protects against the potential side-effects commonly associated with increased testosterone such as prostate stimulation, gynecomastia, or adverse effects on lipid metabolism.

It is known that many estrogens/anti-estrogens/phytoestrogens/SERMs have antitumor effects mediated via estrogen receptor, and they can potentially prevent and treat prostate cancer (S-M. Ho, Estrogens and Anti-Estrogens: Key Mediators of Prostate Carcinogenesis and New Therapeutic Candidates. 91:491-503, 2004).

The SERMs are antiestrogenic in breast and could therefore provide protection against gynecomastia, often associated with testosterone treatments.

The SERMs provide beneficial effects on the lipid profile such as increased HDL, and decreased total cholesterol and LDL. Testosterone is known for instance to decrease HDL, and this adverse effect could thus be counteracted with the SERM.

Both SERMs and testosterone have beneficial effects on bone metabolism by inhibiting bone turnover. Thus, the protective effect of a SERM on bone is likely to be enhanced if it has the ability to increase testosterone.

To sum up, SERMs, particularly the SERMs according to formula (I) presented below, produce the positive response of androgen replacement therapy without the undesired side effects of testosterone, such as adverse effects on prostate or on lipid metabolism, or gynecomastia.

These compounds increase testosterone and thus stimulate muscle growth and reduce subcutaneous and visceral abdominal fat in the treatment of obesity; increase energy and libido and minimize the bone depletion; and have beneficial effects on lipid metabolism.

Preferable SERMs

Suitable selective estrogen receptor modulators (or SERMs) for use in this invention are, for example, the compounds disclosed in V Craig Jordan (2003).

Thus, examples of suitable SERM compounds for use in the present invention are triphenylalkene or triphenylalkane compounds such as compounds disclosed in WO 01/36360, U.S. Pat. No. 4,996,225, U.S. Pat. No. 4,696,949, U.S. Pat. No. 5,750,576 and WO 99/42427 (each incorporated herein by reference) and the toremifene metabolites disclosed in L Kangas (Cancer Chemother Pharmacol 27:8-12, 1990), incorporated herein by reference. As examples of specific drugs disclosed in the aforementioned references can be mentioned toremifene, fispemifene and ospemifene. Droloxifene and iodoxifene also examples of suitable SERMs of triphenylalkene structure.

Other preferable examples of SERM compounds are compounds of benzothiophene structure (described for example in EP 584952, U.S. Pat. No. 4,133,814 and U.S. Pat. No. 4,418,068, each incorporated herein by reference) and arzoxifene.

As further examples of suitable SERMs can be mentioned EM652, EM800, EM776, EM651, EM312, ICI 182780, ERA-923, zindoxifene and deacetylated zindoxifene, ZKl 19010, TSE-4247, lasoxifene and its analogues, particularly those disclosed in EP 802910 (incorporated herein by reference), nafoxidine, basedoxifene, GW5638, GW7604, compound no. 32 disclosed in Jordan (2003), ICI 164384, RU 58668, RU 39411 and EM 319.

Preferably, the SERM is a triphenylalkane compound, a triphenyl-alkene compound, where the alkene chain is halogen-substituted butene or propene, a benzothiophene compound, EM652, EM800, EM776, EM651, EM312, ICI 182780, ERA-923, zindoxifene, deacetylated zindoxifene, ZK119010, TSE-4247, lasoxifene, a lasoxifene analogue, nafoxidine, basedoxifene, GW5638, GW7604, ICI 164384, RU 58668, RU 39411 or EM 319, or an isomer, isomer mixture, metabolite or a pharmaceutically acceptable salt thereof.

Still more preferably, the SERM is a triphenylbutene compound of the formula (I) 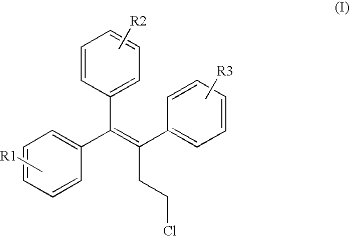
wherein R1 is H, halogen, OCH₃, or OH;

R2 is 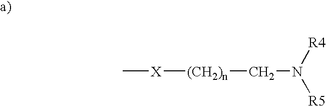
where X is O, NH or S; and n is an integer from 1 to 4; and
R4 and R5, which are the same or different, are a 1 to 4 carbon alky, H, —CH₂C≡CH or —CH₂CH₂OH; or
R4 and R5 form an N-containing five- or six-membered ring or heteroaromatic ring;

b) —Y—(CH₂)ₙCH₂—O—R6
where Y is O, NH or S and n is an integer from 1 to 4; and
R6 is H, —CH₂CH₂OH, or —CH₂CH₂Cl;

or

c) 2,3-dihydroxypropoxy, 2-methylsulfamylethoxy, 2-chloroethoxy, 1-ethyl-2-hydroxy-ethoxy, 2,2-diethyl-2-hydroxyethoxy or carboxymethoxy; and

R3 is H, halogen, OH or —OCH₃

or an isomer, isomer mixture, metabolite or a pharmaceutically acceptable salt thereof.

Still more preferably the SERM is a triphenylbutene compound of the formula (I) the selective estrogen receptor modulator is a triphenylbutene compound of the formula (I) 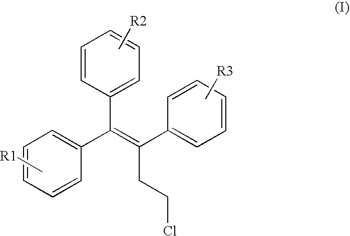
wherein R1 is H, halogen, OCH₃, or OH; and
R2 is

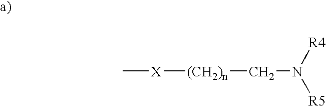

where i) X is NH or S; and n is an integer from 1 to 4; and
R4 and R5, which are the same or different, are a 1 to 4 carbon alkyl, H, —CH₂C≡CH or —CH₂CH₂OH; or
R4 and R5 form an N-containing five- or six-membered ring or heteroaromatic ring;
or

where ii) X is O, and n is an integer from 1 to 4; and
one of R4 and R5 is —CH₂C≡CH or —CH₂CH₂OH and the other is H or a C1-C4-alkyl; or R4 and R5 form an imidazole ring, an N-containing six-membered ring or heteroaromafic ring; or R2 is

b) —Y—(CH₂)ₙCH₂—O—R6

where Y is 0, NH or S and n is an integer from 1 to 4; and
R6 is H, —CH₂CH₂OH, or —CH₂CH₂Cl; or R2 is

c) 2,3-dihydroxypropoxy, 2-methylthioethoxy, 2-chloroethoxy, 1-ethyl-2-hydroxyethoxy, 2,2-diethyl-2-hydroxyethoxy or carboxymethoxy; and

R3 is H, halogen, OH or —OCH₃

or an isomer, isomer mixture, metabolite or a pharmaceutically acceptable salt thereof.

The aforementioned specific SERMs or classes of SERMs are examples only, and other SERMs may be suitable for use in this invention as well.

Preferable SERMs are compounds with tissue specific antiestrogenic or estrogenic effects suitable for men. A preferred SERM is estrogenic in bone and antiestrogenic or mildly estrogenic in other tissues. A classical method to determine the estrogenic profile of a compound is to evaluate estrogenic effect in immature mouse or rat uterus (Terenius L, Acta Endocrinol 66:431-447, 1971). The animals are exposed for 3 days to the compounds to be investigated at the age of 18 days. On the fourth day the animals are sacrificed and body weight and uterine weight is recorded. Estrogens increase the size and weight of the uterus (uterotropic effect) while antiestrogens inhibit this action. The results are given as per cent of estrogen stimulation (100% with estradiol). In our tests, we used a high dose level, i.e. 10-50 mg/kg. Compounds causing an uterotropic effect ≦40% are for this purpose classified as weak estrogenic compounds, compounds causing an uterotropic effect ≧70% are classified as strong estrogenic compounds and compounds in-between, an uterotropic effect of 41-69% are classified as moderate estrogenic agents.

As specific examples of particularly useful SERMs can be mentioned certain compounds of those disclosed in WO 01/36360, namely
(Z)-2-[3-(4-Chloro-1,2-diphenyl-but-1-enyl)phenoxy]ethanol
(Z)-2-{2-[4-(4-Chloro-1,2-diphenylbut-1-enyl)phenoxy]ethoxy}ethanol (also known under the generic name fispemifene)
(Z)-{2-[3-(4-Chloro-1,2-diphenylbut-1-enyl)phenoxy]ethyl}dimethylamine
(E)-3-{4-Chloro-1-[4-(2-hydroxyethoxy)phenyl]-2-phenyl-but-1-enyl}-phenol,
(E)-3-{4-Chloro-1-[4-(2-imidazol-1-yl-ethoxy)phenyl]-2-phenyl-but-1-enyl}-phenol,
(Z)-3-{4-Chloro-1-[4-(2-imidazol-1-yl-ethoxy)phenyl]-2-phenyl-but-1-enyl}-phenol,
and (Z)-2-[4-(4-Chloro-1,2-diphenyl-but-1-enyl)phenoxy]ethanol (ospemifene)

The aforementioned seven compounds are all classified as weak estrogenic SERMs.

For the purpose of this invention, the SERM or its isomer, isomer mixture or their pharmaceutically acceptable salts can be administered by various routes. The suitable administration forms include, for example, oral formulations; parenteral injections including intravenous, intramuscular, intradermal and subcutanous injections; and transdermal or rectal formulations. Suitable oral formulations include e.g. conventional or slow-release tablets and gelatine capsules.

The required dosage of the SERM compounds will vary with the particular condition being treated, the severity of the condition, the duration of the treatment, the administration route and the specific compound being employed. For example, fispemifene can be administered perorally preferentially once daily. The daily dose may be 5-1500 mg, preferably 20-1500 mg. Fispemifene can be given as tablets or other formulations like gelatine capsules alone or mixed in any clinically acceptable non-active ingredients which are used in the pharmaceutical industry.

The invention will be illuminated by the following non-restrictive Experimental Section.

### EXPERIMENTAL SECTION

Methods and Materials

Fispemifene has been studied in two phase I studies in humans—in a single dose and a repeated dose study. Effect of fispemifene on hormone levels was one main focus of the repeated dose study. The phase I repeated dose study (number 101-50202) was a randomized, double-blind, placebo-controlled 28-day dose-escalation study performed in 32 healthy, elderly men, aged 50-68 years. The main objective of the study was to investigate the tolerability, safety and pharmacokinetics of fispemifene after repeated oral doses, but the study focused also on the effects of fispemifene on serum testosterone, estradiol, and other relevant hormones. The fispemifene doses 10, 30, 100 and 300 mg per day and placebo were administered once every morning as capsules containing 10 mg or 100 mg of fispemifene, or placebo. The dose was escalated to the next higher dose level, if the previous dose had been safe and well tolerated evaluated by the laboratory safety determinations and ultrasound of mammagy glands.

The variables for safety and tolerability were adverse events, vital signs, 12-lead ECG, clinical laboratory evaluations, physical examination, ultrasound examinations (mammary glands) and inhibin b. For pharmacokinetics, the concentrations of fispemifene and its metabolite(s) were to be evaluated. For pharmacodynamics, serum concentrations of FSH, LH, estradiol, testosterone, SHBG, prolactin, aldosterone, cortisol and TSH before and during treatment were measured and compared with the concentrations in the placebo-group.

Results on the Effects of Fispemifene on Hormones

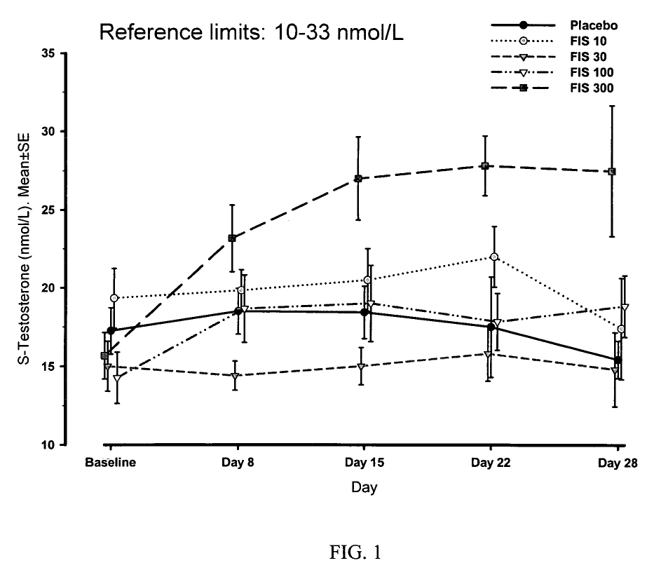

Surprisingly, fispemifene increased the serum concentrations of testosterone, FSH, LH, and SHBG (Table 1) during the 28 days of treatment. Testosterone was increased statistically significantly with 100 mg and 300 mg fispemifene compared with placebo. With the 300 mg dose, the increase in the mean total testosterone was about 75% compared to the baseline concentration. Two out of six men treated with the highest fispemifene dose had their serum testosterone level above the upper limit of normal range (i.e., 33 nmol/L) during treatment. The rest two had a significant increase within the reference range. All the six men had normal testosterone value at baseline. With the 100 mg dose, the increase of the mean total testosterone was about 32%, and all the six men in the group had their testosterone level increased within the reference range. The increase in total testosterone levels in serum is illustrated by group in FIG. 1. There were no safety concerns raised with any dose suggesting that even a higher dose could be utilized if deemed appropriate.

TABLE 1
Serum total testosterone concentrations (mean and SD) and the other hormones
at baseline and during treatment in the fispemifene study 101-50202 by dose.

| | | | Fispemifene | | Fispemifene | | Fispemifene | | Fispemifene | |
|---|---|---|---|---|---|---|---|---|---|---|
| | Placebo | | 10 mg | | 30 mg | | 100 mg | | 300 mg | |
| | Mean | SD | Mean | SD | Mean | SD | Mean | SD | Mean | SD |
| Testosterone | | | | | | | | | | |
| (nmol/l) | | | | | | | | | | |
| Baseline | 17.25 | 4.2 | 19.33 | 4.7 | 15.00 | 3.5 | 14.27 | 4.0 | 15.67 | 3.6 |
| Day 8 | 18.50 | 4.1 | 19.83 | 3.3 | 14.40 | 2.1 | 18.67 | 5.3 | 23.17 | 5.2 |
| Day 15 | 18.43 | 4.4 | 20.50 | 4.9 | 15.00 | 2.7 | 19.00 | 6.0 | 27.00 | 6.5 |
| Day 22 | 17.50 | 8.5 | 22.00 | 4.4 | 15.80 | 3.9 | 17.83 | 4.5 | 27.83 | 4.7 |
| Day 28 | 15.43 | 3.2 | 17.40 | 7.2 | 14.80 | 5.3 | 18.83 | 4.8 | 27.50 | 10.3 |
| FSH (U/l) | | | | | | | | | | |
| Baseline | 5.60 | 3.4 | 5.42 | 3.6 | 9.14 | 13.4 | 6.30 | 5.6 | 6.80 | 5.4 |
| Day 8 | 5.65 | 2.9 | 5.87 | 4.2 | 9.78 | 14.2 | 7.68 | 7.8 | 8.80 | 7.6 |
| Day 15 | 4.67 | 1.6 | 5.20 | 2.9 | 10.14 | 14.6 | 8.10 | 9.0 | 8.73 | 7.2 |
| Day 22 | 4.47 | 1.6 | 6.60 | 4.1 | 10.18 | 15.1 | 8.20 | 9.0 | 8.85 | 8.1 |
| Day 28 | 4.29 | 1.7 | 5.66 | 3.7 | 8.42 | 11.6 | 7.73 | 7.9 | 7.57 | 7.0 |
| LH (U/l) | | | | | | | | | | |
| Baseline | 3.11 | 1.6 | 3.47 | 1.0 | 3.58 | 2.0 | 4.12 | 1.9 | 4.58 | 2.7 |
| Day 8 | 3.29 | 0.8 | 3.12 | 1.5 | 4.26 | 2.2 | 5.52 | 4.2 | 6.80 | 3.5 |
| Day 15 | 3.31 | 0.9 | 2.87 | 1.1 | 5.02 | 2.4 | 6.82 | 7.5 | 6.75 | 4.6 |
| Day 22 | 2.80 | 0.8 | 3.56 | 1.2 | 4.32 | 2.3 | 7.18 | 8.3 | 7.77 | 6.6 |
| Day 28 | 2.71 | 0.9 | 3.02 | 0.9 | 4.42 | 2.0 | 7.60 | 9.6 | 6.70 | 4.8 |
| Estradiol | | | | | | | | | | |
| (pmol/l) | | | | | | | | | | |
| Baseline | 100.6 | 31.2 | 106.2 | 20.9 | 97.8 | 17.9 | 84.3 | 22.6 | 102.5 | 30.0 |
| Day 8 | 93.8 | 17.1 | 94.7 | 31.2 | 105.6 | 29.8 | 108.3 | 28.9 | 104.0 | 20.0 |
| Day 15 | 85.0 | 31.6 | 81.7 | 25.4 | 102.4 | 22.2 | 111.5 | 48.2 | 97.8 | 26.9 |
| Day 22 | 75.0 | 32.4 | 116.6 | 15.1 | 99.6 | 20.4 | 106.3 | 37.4 | 95.5 | 32.9 |
| Day 28 | 73.6 | 32.6 | 75.0 | 20.1 | 87.0 | 22.2 | 94.5 | 48.4 | 89.7 | 30.9 |
| SHBG | | | | | | | | | | |
| (nmol/l) | | | | | | | | | | |
| Baseline | 49.1 | 18.6 | 47.7 | 19.9 | 34.2 | 12.8 | 41.7 | 29.4 | 50.7 | 15.1 |
| Day 8 | 44.5 | 16.1 | 46.3 | 21.1 | 34.2 | 12.2 | 47.7 | 35.2 | 64.2 | 21.3 |
| Day 15 | 46.0 | 19.1 | 48.2 | 22.8 | 37.4 | 20.8 | 52.0 | 39.5 | 66.2 | 21.1 |
| Day 22 | 44.9 | 18.4 | 50.2 | 27.1 | 37.2 | 19.2 | 55.7 | 45.3 | 65.2 | 14.8 |
| Day 28 | 45.0 | 18.5 | 45.2 | 24.3 | 36.6 | 19.1 | 50.8 | 42.8 | 58.3 | 12.3 |

Discussion and Conclusions

Fispemifene induced a clinically and statistically significant and dose dependent increase in the serum testosterone concentration within 28 days from the start of the treatment. Also, within the 28-day treatment, the increase in testosterone serum concentration was seen in all the subjects treated with 100 mg or 300 mg fispemifene. An increase of 75% from baseline can be considered clinically highly significant, and thus clinical benefits in men with low testosterone can be expected. The increases also in LH and FSH suggest that fispemifene has an antiestrogenic effect on hypothalamus/hypophysis, and that the increase in testosterone occurs due to the increase in the hypophyseal hormones. The increase in testosterone is moderate and, therefore, no harmful effects often associated with external testosterone administration are expected. Furthermore, a SERM is likely to provide protection against the possible safety problems of testosterone like development of prostate cancer. Thus, a SERM increasing testosterone provides an optimal treatment for hypogonadism, balancing the efficacy and safety of the increased testosterone.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

1. Method for the treatment or prevention of androgen deficiency in a male individual, said method comprising administering to the individual an effective amount of a selective estrogen receptor modulator, or an isomer, isomer mixture, metabolite or a pharmaceutically acceptable salt thereof.

2. The method according to claim 1, wherein the selective estrogen receptor modulator is a triphenylalkane compound, a triphenyl-alkene compound, where the alkene chain is halogen-substituted butene or propene, a benzothiophene compound, EM652, EM800, EM776, EM651, EM312, ICI 182780, ERA-923, zindoxifene, deacetylated zindoxifene, ZK119010, TSE-4247, lasoxifene, a lasoxifene analogue, nafoxidine, basedoxifene, GW5638, GW7604, ICI 164384, RU 58668, RU 39411 or EM 319, or an isomer, isomer mixture, metabolite or a pharmaceutically acceptable salt thereof.

3. The method according to claim 2, wherein the selective estrogen receptor modulator is a triphenylbutene compound of the formula (I) 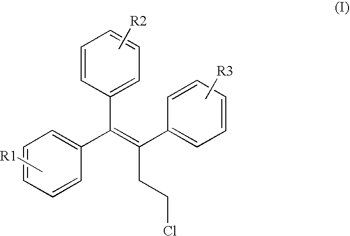
wherein R1 is H, halogen, OCH₃, or OH; and
R2 is 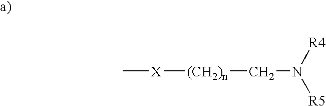
where X is O, NH or S; and n is an integer from 1 to 4; and
R4 and R5, which are the same or different, are a 1 to 4 carbon alkyl, H, —CH₂C≡CH or —CH₂CH₂OH; or
R4 and R5 form an N-containing five- or six-membered ring or heteroaromatic ring;
b) —Y—(CH₂)ₙCH₂—O—R6
where Y is O, NH or S and n is an integer from 1 to 4; and
R6 is H, —CH₂CH₂OH, or —CH₂CH₂Cl; or
c) 2,3-dihydroxypropoxy, 2-methylsulfamylethoxy, 2-chloroethoxy, 1-ethyl-2-hydroxyethoxy, 2,2-diethyl-2-hydroxyethoxy or carboxymethoxy; and
R3 is H, halogen, OH or —OCH₃ or
an isomer, isomer mixture, metabolite or a pharmaceutically acceptable salt thereof.

4. The method according to claim 2, wherein the selective estrogen receptor modulator is a triphenylbutene compound of the formula (I) 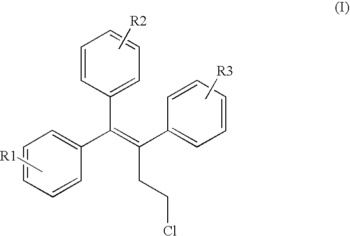
wherein R1 is H, halogen, OCH₃, or OH; and
R2 is 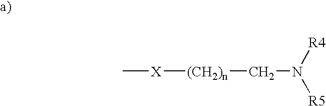
where
i) X is NH or S; and n is an integer from 1 to 4; and
R4 and R5, which are the same or different, are a 1 to 4 carbon alkyl, H, —CH₂C≡CH or —CH₂CH₂OH; or
R4 and R5 form an N-containing five- or six-membered ring or heteroaromatic ring; or
ii) X is O, and n is an integer from 1 to 4; and
one of R4 and R5 is —CH₂C≡CH or —CH₂CH₂OH and the other is H or a C1-C4-alkyl; or R4 and R5 form an imidazole ring, an N-containing six-membered ring or heteroaromatic ring;
b)—Y—(CH₂)ₙCH₂—O—R6
where Y is O, NH or S and n is an integer from 1 to 4; and
R6 is H, —CH₂CH₂OH, or —CH₂CH₂Cl; or
c) 2,3-dihydroxypropoxy, 2-methylthioethoxy, 2-chloroethoxy, 1-ethyl-2-hydroxyethoxy, 2,2-diethyl-2-hydroxyethoxy or carboxymethoxy; and
R3 is H, halogen, OH or —OCH₃ or
an isomer, isomer mixture, metabolite or a pharmaceutically acceptable salt thereof.

5. The method according to claim 1, wherein the selective estrogen receptor modulator is a compound with tissue specific antiestrogenic or estrogenic effects suitable for men.

6. The method according to claim 5, wherein the selective estrogen receptor modulator is selected from the group consisting of
(Z)-2-[3-(4-Chloro-1,2-diphenyl-but-1-enyl)phenoxy]ethanol,
(Z)-2-{2-[4-(4-Chloro-1,2-diphenylbut-1-enyl)phenoxy]ethoxy}ethanol (fispemifene),
(Z)-{2-[3-(4-Chloro-1,2-diphenylbut-1-enyl)phenoxy]ethyl}dimethylamine,
(E)-3-{4-Chloro-1-[4-(2-hydroxyethoxy)phenyl]-2-phenyl-but-1-enyl}-phenol,
(E)-3-{4-Chloro-1-[4-(2-imidazol-1-yl-ethoxy)phenyl]-2-phenyl-but-1-enyl}-phenol,
(Z)-3-{4-Chloro-1-[4-(2-imidazol-1-yl-ethoxy)phenyl]-2-phenyl-but-1-enyl}-phenol, and
(Z)-2-[4-(4-chloro-1,2-diphenyl-but-1-enyl)phenoxy]ethanol (ospemifene),
or an isomer, isomer mixture, metabolite or a pharmaceutically acceptable salt thereof.

7. The method according to claim 6, wherein the selective estrogen receptor modulator is fispemifene or metabolite or a pharmaceutically acceptable salt thereof.

8. A method for prevention or treatment of a disease or disorder in a male individual, said disease or disorder being caused by androgen deficiency in said individual, said method comprising administering to the individual an effective amount of a selective estrogen receptor modulator as defined in claim 1, or an isomer, isomer mixture, metabolite or a pharmaceutically acceptable salt thereof.

9. The method according to claim 8, wherein said disease or disorder is selected from the group consisting of
hypogonadism, particularly but not restricted to secondary hypogonadism resulting from disease or disorders such as Kallman's Syndrome, Prader-Labhart-Willi's Syndrome, Laurence-Moon-Biedl's Syndrome, pituitary insufficiency/adenomas, Pasqualini's Syndrome, hemochromatosis, hyperprolactinemia, pituitary-hypothalamic injury from tumors, trauma, radiation, obesity, chronic illness, such as diabetes mellitus, hypotyroidism or other disease or disorder that may affect central production of gonadotropin;
age-related testosterone deficiency and diseases or disorders resulting therefrom, such as impaired muscle strength, sexual dysfunction, decreased libido, loss of muscle mass, decreased bone density, depressed mood, and decreased cognitive function; and
any muscular atrophy/dystrophies; lipodistrophy; long-term critical illness; sarcopenia; frailty or age-related functional decline; reduced muscle strength and function; muscle wasting from HIV; chronic renal failure, reduced bone density or growth; catabolic side effects of glucocorticoids; chronic fatigue syndrome; reduced bone fracture repair; acute fatigue syndrome and muscle loss following elective surgery; cachesia; chronic catabolic state; eating disorders; side effects of chemotherapy; wasting; depression; nervousness irritability; stress; growth retardation; senescence outfall symptoms; reduced cognitive function; anaemia; male contraception; infertility; Syndrome X; diabetic complications or obesity.

10. The method according to claim 9, wherein said disease or disorder is selected from the group consisting of hypogonadism, particularly but not restricted to secondary hypogonadism and diseases or disorders resulting therefrom and age-related testosterone deficiency and diseases or disorders resulting therefrom, and said selective estrogen receptor modulator is a triphenylbutene compound of the formula (I) 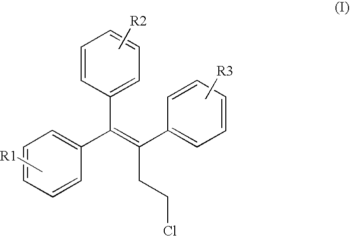
wherein R1 is H, halogen, OCH₃, or OH; and
R2 is 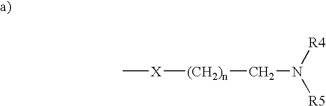
where X is O, NH or S; and n is an integer from 1 to 4; and
R4 and R5, which are the same or different, are a 1 to 4 carbon alkyl, H, —CH₂C≡CH or —CH₂CH₂OH; or
R4 and R5 form an N-containing five- or six-membered ring or heteroaromatic ring;
b) —Y—(CH₂)ₙCH₂—O—R6
where Y is O, NH or S and n is an integer from 1 to 4; and
R6 is H, —CH₂CH₂OH, or —CH₂CH₂Cl; or
c) 2,3-dihydroxypropoxy, 2-methylsulfamylethoxy, 2-chloroethoxy, 1-ethyl-2-hydroxyethoxy, 2,2-diethyl-2-hydroxyethoxy or carboxymethoxy; and
R3 is H, halogen, OH or —OCH₃ or
an isomer, isomer mixture, metabolite or a pharmaceutically acceptable salt thereof.

11. The method according to claim 10, wherein said selective estrogen receptor modulator is a triphenylbutene compound of the formula (I) 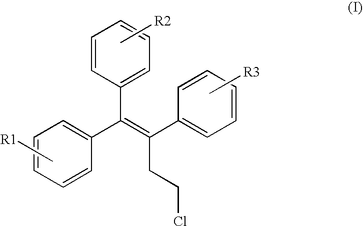
wherein R1 is H, halogen, OCH₃, or OH; and
R2 is 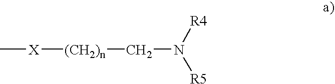
where
i) X is NH or S; and n is an integer from 1 to 4; and
R4 and R5, which are the same or different, are a 1 to 4 carbon alkyl, H, —CH₂C≡CH or —CH₂CH₂OH; or
R4 and R5 form an N-containing five- or six-membered ring or heteroaromatic ring; or
ii) X is O, and n is an integer from 1 to 4; and
one of R4 and R5 is —CH₂C≡CH or —CH₂CH₂OH and the other is
H or a C1-C4-alkyl; or R4 and R5 form an imidazole ring, an N-containing six-membered ring or heteroaromatic ring; or R2 is
b) —Y—(CH₂)ₙCH₂—O—R6
where Y is O, NH or S and n is an integer from 1 to 4; and
R6 is H, —CH₂CH₂OH, or —CH₂CH₂Cl; or
c) 2,3-dihydroxypropoxy, 2-methylthioethoxy, 2-chloroethoxy, 1-ethyl-2-hydroxyethoxy, 2,2-diethyl-2-hydroxyethoxy or carboxymethoxy; and
R3 is H, halogen, OH or —OCH₃ or
an isomer, isomer mixture, metabolite or a pharmaceutically acceptable salt thereof.

12. The method according to claim 11, wherein the selective estrogen receptor modulator is selected from the group consisting of
(Z)-2-[3-(4-Chloro-1,2-diphenyl-but-1-enyl)phenoxy]ethanol,
(Z)-2-{2-[4-(4-Chloro-1,2-diphenylbut-1-enyl)phenoxy]ethoxy}ethanol (fispemifene),
(Z)-{2-[3-(4-Chloro-1,2-diphenylbut-1-enyl)phenoxy]ethyl}dimethylamine,
(E)-3-{4-Chloro-1-[4-(2-hydroxyethoxy)phenyl]-2-phenyl-but-1-enyl}-phenol,
(E)-3-{4-Chloro-1-[4-(2-imidazol-1-yl-ethoxy)phenyl]-2-phenyl-but-1-enyl}-phenol,
(Z)-3-{4-Chloro-1-[4-(2-imidazol-1-yl-ethoxy)phenyl]-2-phenyl-but-1-enyl}-phenol, and
(Z)-2-[4-(4-chloro-1,2-diphenyl-but-1-enyl)phenoxy]ethanol (ospemifene),
or an isomer, isomer mixture, metabolite or a pharmaceutically acceptable salt thereof.

13. The method according to claim 12 wherein the selective estrogen receptor modulator is fispemifene or an isomer, isomer mixture, metabolite or a pharmaceutically acceptable salt thereof.
